(19) United States
(12) Patent Application Publication
(10) Pub. No.: US 2022/0280387 A1
(43) Pub. Date: Sep. 8, 2022

(54) DOWN STREAMING PROCESS FOR THE PRODUCTION OF POLYUNSATURATED FATTY ACID SALTS

(71) Applicant: Evonik Operations GmbH, Essen (DE)

(72) Inventors: Ashish GUHA, Mumbai (IN); Theresia KUNTZ, Frankfurt (DE); Andreas EMRICH, Mainz (DE); Christian MAHLMEISTER, Ginsheim-Gustavsburg (DE); Johanna PETERS, Frankfurt (DE); Milan LATINOVIC (Deceased), (US); Günter KNAUP, Hanau (DE); Joerg LOTZ, Kalbach (DE); Thomas DIEHL, Floersheim am Main (DE); Vinay JAIN, Mumbai (IN); Eduard HARTMANN, Rodenbach (DE)

(73) Assignee: Evonik Operations GmbH, Essen (DE)

(21) Appl. No.: 17/633,460

(22) PCT Filed: Aug. 7, 2020
(86) PCT No.: PCT/EP2020/072213

§ 371 Date: Feb. 7, 2022

(30) Foreign Application Priority Data
Aug. 8, 2019 (IN) 201941032090
Oct. 10, 2019 (EP) 19202421.4

(51) Int. Cl. A61J 3/10; A23L 33/12; B01J 2/02; A61K 9/20

(52) U.S. Cl. CPC A61J 3/10; A23L 33/12; B01J 2/02; A61K 9/2013; A61K 9/2009

(57) ABSTRACT

The invention provides an improved down streaming process for production of polyunsaturated fatty acid salts suitable for tableting by direct compression.

The invention provides an improved down streaming process for production of polyunsaturated fatty acid salts suitable for tableting by direct compression.

Polyunsaturated fatty acids (PUFAs), such as omega-3 fatty acids, particularly eicosapentaenoic acid (EPA) and docosahexaenoic acid (DHA), are linked to numerous positive health effects on the cardiovascular system, on inflammatory disorders, on brain development and function, on disruptions of the central nervous system and on other areas (C. H. S. Ruxton, S. C. Reed, M. J. A. Simpson, K. J. Millington, J. Hum. Nutr. Dietet 2004, 17, 449). Therefore, the intake of omega-3 fatty acids is supported by statements of regulatory agencies. For instance, the EFSA (European Food Safety Authority) recommends for adults a daily intake of 250 mg of EPA+DHA (EFSA Panel on Dietetic Products, Nutrition and Allergies, EFSA Journal 2010, 8 (3), 1461). The AHA (American Heart Association) advises the intake of at least two meals of fatty fish per week for persons without documented cardiovascular disorders, the intake of about 1 g of EPA+DHA per day from fish or food supplements for persons with documented cardiovascular disorders and the intake of 2-4 g of EPA+DHA per day for the treatment of raised blood lipid values (P. M. Kris-Etherton, W. S. Harris, L. J. Appel, Circulation 2002, 106, 2747). Moreover, the authorities have expressly approved health claims for omega-3 fatty acids determined on the basis of clinical studies (EU Register on Nutrition and Health Claims; see also: EFSA Journal 2011, 9 (4), 2078). Therefore, omega-3 fatty acids, especially from fish oil but also from other plant or microbial sources, are increasingly used as food supplements, food additives and medicaments.

According to standard nomenclature, polyunsaturated fatty acids are classified according to the number and position of the double bonds. There are two series or families, depending on the position of the double bond which is closest to the methyl end of the fatty acid. The omega-3 series comprises a double bond at the third carbon atom whereas the omega-6 series has no double bond up to the sixth carbon atom. Thus, docosahexaenoic acid (DHA) has a chain length of 22 carbon atoms with 6 double bonds beginning with the third carbon atom from the methyl end and is referred to as “22:6 n-3” (all-cis-4,7,10,13,16,19-docosahexaenoic acid). Another important omega-3 fatty acid is eicosapentaenoic acid (EPA), which is referred to as “20:5 n-3” (all-cis-5,8,11,14,17-eicosapentaenoic acid).

Most of the omega-3 fatty acid products introduced to the market are offered in the form of oils, starting from fish oil with a content of about 30% omega-3 fatty acids up to concentrates with over 90% content of EPA or DHA or mixtures of these two omega-3 fatty acids. The formulations used are predominantly soft gelatine capsules. In addition, numerous further product forms have been described, such as microencapsulations or powder preparations (C. J. Barrow, B. Wang, B. Adhikari, H. Liu, Spray drying and encapsulation of omega-3 oils, in: Food enrichment with omega-3 fatty acids (Eds.: C. Jacobsen, N. S. Nielsen, A. Frisenfeldt Horn, A. -D. Moltke Soerensen), pp. 194-225, Woodhead Publishing Ltd., Cambridge 2013, ISBN 978-0-85709-428-5; T. -L. Torgersen, J. Klaveness, A. H. Myrset, US 2012/0156296 A1). Chemically, these are usually triglycerides or fatty acid ethyl esters with various concentrations of omega-3 fatty acids, while phospholipids, e.g. as krill oil, free fatty acids (T. J. Maines, B. N. M. Machielse, B. M. Mehta, G. L. Wisler, M. H. Davidson, P. R. Wood, US 2013/0209556 A1; M. H. Davidson, G. H. Wisler, US 2013/0095179 A1; N. J. Duragkar, US 2014/0018558 A1; N. J. Duragkar, US 2014/0051877 A1) and various salts of fatty acids are also known, e.g. with potassium, sodium, ammonium (H. J. Hsu, S. Trusovs, T. Popova, US 8203013 B2), calcium and magnesium, (J. A. Kralovec, H. S. Ewart, J. H. D. Wright, L. V. Watson, D. Dennis, C. J. Barrow, J. Functional Foods 2009, 1, 217; G. K. Strohmaier, N. D. Luchini, M. A. Varcho, E. D. Frederiksen, U.S. Pat. No. 7,098,352 B2), where these salts are not water-soluble, aminoalcohols (P. Rongved, J. Klaveness, US 2007/0213298 A1), amine compounds such as piperazine (B. L. Mylari, F. C. Sciavolino, US 2014/0011814 A1), and guanidine compounds such as metformin (M. Manku, J. Rowe, US 2012/0093922 A1; B. L. Mylari, F. C. Sciavolino, US 2012/0178813 A1; B. L. Mylari, F. C. Sciavolino, US 2013/0281535 A1; B. L. Mylari, F. C. Sciavolino, WO 2014/011895 A2). The bioavailability of the different omega-3 derivatives for the human body is very diverse. Since omega-3 fatty acids as free fatty acids together with monoacyl glycerides are absorbed in the small intestine, the bioavailability of free omega-3 fatty acids is better than that of triglycerides or ethyl esters since these have firstly to be cleaved to the free fatty acids in the digestive tract (J. P. Schuchhardt, A. Hahn, Prostaglandins Leukotrienes Essent. Fatty Acids 2013, 89, 1). The stability to oxidation is also very different in different omega-3 derivatives. Free omega-3 fatty acids are described as very sensitive to oxidation (J. P. Schuchhardt, A. Hahn, Prostaglandins Leukotrienes Essent. Fatty Acids 2013, 89, 1). For the use of a solid omega-3 form, an increased stability compared to liquid products is assumed (J. A. Kralovec, H. S. Ewart, J. H. D. Wright, L. V. Watson, D. Dennis, C. J. Barrow, J. Functional Foods 2009, 1, 217).

Furthermore, preparations of omega-3 fatty acids with diverse amino acids, such as lysine and arginine, are known, either as mixtures (P. Literati Nagy, M. Boros, J. Szilbereky, I. Racz, G. Soos, M. Koller, A. Pinter, G. Nemeth, DE 3907649 Al) or as salts (B. L. Mylari, F. C. Sciavolino, WO 2014/011895 Al; T. Bruzzese, EP 0699437 Al; T. Bruzzese, EP0734373 B1; T. Bruzzese, U.S. Pat. No. 5,750,572, J. Torras et al., Nephron 1994, 67, 66; J. Torras et al., Nephron 1995, 69, 318; J. Torras et al., Transplantation Proc. 1992, 24 (6), 2583; S. El Boustani et al., Lipids 1987, 22 (10), 711; H. Shibuya, US 2003/0100610 A1). The preparation of omega-3 aminoalcohol salts by spray-drying is also mentioned (P. Rongved, J. Klaveness, US 2007/0213298 A1).

EP 0734373 B1 describes the preparation of DHA amino acid salts by evaporation to dryness under high vacuum and low temperature or freeze-drying. The resulting products are described as very thick, transparent oils which transform at low temperature into solids of waxy appearance and consistency. Although a tableting formulation has also been mentioned with the use of significant amount of adsorbing diluents, using such oily substance for tableting at larger scales poses significant processing challenges. Moreover, the consistency of such tablets at different temperatures of storage could be altered.

WO 2016/102323 A1 and WO2016/102316 A1 disclose processes for increasing the stability of a composition comprising polyunsaturated omega-3 fatty acids or omega-6 fatty acids against oxidation. The processes comprise the following steps: (i) providing a starting composition comprising at least one polyunsaturated omega-3 or omega-6 fatty acid component; (ii) providing a lysine composition; (iii) admixing aqueous, aqueous-alcoholic or alcoholic solutions of starting composition and lysine composition, and subjecting resulting admixture to spray drying conditions subsequently, thus forming a solid product composition comprising at least one salt of a cation derived from lysine with an anion derived from a polyunsaturated omega-3 or omega-6 fatty acid. Although in this invention a useful process for production of solid PUFA salt of amino acid is described using spray drying conditions, the powder obtained at the end lacks useful properties necessary for production of dosage forms like tablets.

Problem: PUFA amino acid salts are known in prior art, and processes for preparing the same are also disclosed. However, to make these powders suitable for tableting, especially on commercial scale machines, it's critical to manage the powder characteristics to optimal.

It was observed that in order to prepare a powder (of omega amino acid salts) suitable for tableting application, one or more additional down streaming processes like granulation, drying and sizing are required, which is not desirable from costs and industrial applicability point of view. It is required to develop a single step downstreaming process for drying and granulation together while also producing an omega amino acid salt powder suitable for tableting.

Solution: It was found that by using the spray granulation process as a downstreaming process in the production of PUFA amino acid salt solid powder, against pure spray drying, it can provide exceptionally good powder properties well suited for tableting. Additionally, it was also found that a specific substantially monomodal particle size distribution or a bimodal distribution with certain characteristics in the PSD curve is of particular advantage. Some of the scale-independent process parameters were found necessary for producing the optimal powder characteristics. Further adaptations/modifications/improvements of the spray granulation process, such as continuous spray granulation, and top spray batch granulation processes work equally well.

The documents WO 2016/102323 A1 and WO2016/102316 A1 disclose spray-drying conditions for the stabilization of PUFAs against oxidation. Spray drying conditions according to the present invention comprise pure spray drying, where a dry powder is produced from a liquid or slurry by rapidly drying with hot gas and spray granulation, where free-flowing granulates are produced from liquids, after a spray drying step. With a spray granulation process, the product properties can be varied in many ways by setting process technical parameters and configurations.

Spray granulation in the fluidized bed permits liquids to be directly made into free-flowing granulate with specific product properties. Liquids containing solids, such as solutions, suspensions or melts, are sprayed into a fluidized bed system. Due to the high heat exchange the aqueous or organic solutions evaporate immediately, and the solids form small particles as starter cores. These are sprayed with other liquids which in turn, after evaporation, form a hard coating around the starter core. This step is continuously repeated in the fluidized bed so that the granulate grows layer by layer like an onion. Alternatively, a defined volume of suitable starter cores can be provided. In this option, the liquid only serves as a vehicle for the solids that are being applied.

This process variant is often used in a continuous fluidized bed system with air-classifying discharge. Through the continuous removal of the finished granules from the drying room, the amount of particles in the fluidized bed remains constant.

The granules can be very dense because they have grown in layers and are thus resistant to abrasion. Parameters such as particle size, residual moisture and solids content can be specifically adapted to achieve the most varying product properties. Using spray granulation, medium-sized particles of 50 micrometers to 5 millimeters can be produced. Properties such as ability to flow, not abrade, not flake, easily dissolve or be optimally dosed can be imparted to solids using spray granulation. The dust-free granules have a dense surface structure and high bulk density and are low hygroscopic because of their small surface. The optimal solution for converting liquid substances into a solid product form.

In the context of the present invention the term PUFA is used interchangeably with the term polyunsaturated fatty acid and defined as follows: Fatty acids are classified based on the length and saturation characteristics of the carbon chain. Short chain fatty acids have 2 to about 6 carbons and are typically saturated. Medium chain fatty acids have from about 6 to about 14 carbons and are also typically saturated. Long chain fatty acids have from 16 to 24 or more carbons and may be saturated or unsaturated. In longer chain fatty acids there may be one or more points of unsaturation, giving rise to the terms “monounsaturated” and “polyunsaturated,” respectively. In the context of the present invention long chain polyunsaturated fatty acids having 20 or more carbon atoms are designated as polyunsaturated fatty acids or PUFAs.

PUFAs are categorized according to the number and position of double bonds in the fatty acids according to well established nomenclature. There are two main series or families of LC-PUFAs, depending on the position of the double bond closest to the methyl end of the fatty acid: The omega-3 series contains a double bond at the third carbon, while the omega-6 series has no double bond until the sixth carbon. Thus, docosahexaenoic acid (DHA) has a chain length of 22 carbons with 6 double bonds beginning with the third carbon from the methyl end and is designated “22:6 n-3” (all-cis-4,7,10,13,16,19-docosahexaenoic acid). Another important omega-3 PUFA is eicosapentaenoic acid (EPA) which is designated “20:5 n-3” (all-cis-5,8,11,14,17-eicosapentaenoic acid). An important omega-6 PUFA is arachidonic acid (ARA) which is designated “20:4 n-6” (all-cis-5,8,11,14-eicosatetraenoic acid).

Other omega-3 PUFAs include: Eicosatrienoic acid (ETE) 20:3 (n-3) (all-cis-11,14,17-eicosatrienoic acid), Eicosatetraenoic acid (ETA) 20:4 (n-3) (all-cis-8,11,14,17-eicosatetraenoic acid), Heneicosapentaenoic acid (HPA) 21:5 (n-3) (all-cis-6,9,12,15,18-heneicosapentaenoic acid),

Docosapentaenoic acid (Clupanodonic acid) (DPA) 22:5 (n-3) (all-cis-7,10,13,16,19-docosapentaenoic acid), Tetracosapentaenoic acid 24:5 (n-3) (all-cis-9,12,15,18,21-tetracosapentaenoic acid), Tetracosahexaenoic acid (Nisinic acid) 24:6 (n-3) (all-cis-6,9,12,15,18,21-tetracosahexaenoic acid).

Other omega-6 PUFAs include: Eicosadienoic acid 20:2 (n-6) (all-cis-11,14-eicosadienoic acid), Dihomo-gamma-linolenic acid (DGLA) 20:3 (n-6) (all-cis-8,11,14-eicosatrienoic acid), Docosadienoic acid 22:2 (n-6) (all-cis-13,16-docosadienoic acid), Adrenic acid 22:4 (n-6) (all-cis-7,10,13,16-docosatetraenoic acid), Docosapentaenoic acid (Osbond acid) 22:5 (n-6) (all-cis-4,7,10,13,16-docosapentaenoic acid), Tetracosatetraenoic acid 24:4 (n-6) (all-cis-9,12,15,18-tetracosatetraenoic acid), Tetracosapentaenoic acid 24:5 (n-6) (all-cis-6,9,12,15,18-tetracosapentaenoic acid).

Preferred omega-3 PUFAs used in the embodiments of the present invention are docosahexaenoic acid (DHA) and eicosapentaenoic acid (EPA).

Compositions comprising polyunsaturated omega-3 or omega-6 fatty acids that can be used for the process of the present invention may be any compositions containing substantial amounts of free polyunsaturated omega-3 or omega-6 fatty acids. Such compositions may further comprise other naturally occurring fatty acids in free form. In addition, such compositions may further comprise constituents that by themselves are solid, liquid or gaseous at room temperature and standard atmospheric pressure. Corresponding liquid constituents include constituents that can easily be removed by evaporation and could thus be considered as volatile constituents as well as constituents that are difficult to remove by evaporation and could thus be considered as non-volatile constituents. In the present context gaseous constituents are considered as volatile constituents. Typical volatile constituents are water, alcohols and supercritical carbon dioxide.

Compositions comprising polyunsaturated omega-3 or omega-6 fatty acids that can be used for the process of the present invention may be obtained from any suitable source material which, additionally, may have been processed by any suitable method of processing such source material. Typical source materials include any part of fish carcass, vegetables and other plants as well as material derived from microbial and/or algal fermentation. Typically, such material further contains substantial amounts of other naturally occurring fatty acids. Typical methods of processing such source materials may include steps for obtaining crude oils such as extraction and separation of the source material, as well as steps for refining crude oils such as settling and degumming, de-acidification, bleaching, and deodorization, and further steps for producing omega-3 or omega-6 PUFA-concentrates from refined oils such as de-acidification, trans-esterification, concentration, and deodorization (cf. e.g. EFSA Scientific Opinion on Fish oil for Human Consumption). Any processing of source materials may further include steps for at least partially transforming omega-3 or omega-6 PUFA-esters into the corresponding free omega-3 or omega-6 PUFAs or inorganic salts thereof.

Preferred compositions comprising polyunsaturated omega-3 or omega-6 fatty acids used for the process of the present invention can be obtained from compositions mainly consisting of esters of omega-3 or omega-6 PUFAs and other naturally occurring fatty acids by cleavage of the ester bonds and subsequent removal of the alcohols previously bound as esters. Preferably, ester cleavage is performed under basic conditions. Methods for ester cleavage are well known in the art.

The present invention is directed to a process for granulating a polyunsaturated fatty acid salt, comprising the steps of:
i. providing a starting composition comprising at least one polyunsaturated omega-3 or omega-6 fatty acid component;
ii. providing a counter ion composition;
iii. admixing aqueous, aqueous-alcoholic or alcoholic solutions of starting composition and counter ion composition,
iv. and subjecting resulting admixture to spray granulation in a fluidized bed subsequently, thus forming a solid product composition comprising at least one salt of a cation derived from the counter ion with an anion derived from a polyunsaturated omega-3 or omega-6 fatty acid;
   wherein the counter ion composition is provided in such manner that the ratio of the amount of carboxylic acid functions in the starting composition provided in step (i) and the amount of counter ions provided in step (ii) is in a range of 1:0.5 to 1:2 (carboxylic acid functions:counter ions) on molar basis.

According to the present invention the counter ion composition is provided in such manner that the ratio of the amount of carboxylic acid functions in the starting composition provided in step (i) and the amount of counter ions provided in step (ii) is in a range of 1:0.5 to 1:2 (carboxylic acid functions:counter ions) on molar basis. In other words, this means that the starting omega-3 or omega-6 fatty acid component and the counter ion composition shall be provided in equimolar quantities to facilitate quantitative salt formation.

In a preferred embodiment, the counter ion composition in step (ii) is provided in such a manner that the ratio R=n(ca)/n(ci) of the amount of carboxylic acid functions n(ca) in the starting composition provided in step (i) and the total amount of free counter ion n(ci) in the counter ion composition provided in step (ii) is in a range selected from 0.9<R<1.1, 0.95<R<1.05, 0.98<R<1.02. In a particularly preferred embodiment R is in the range 0.98<R<1.02. The amount of carboxylic acid functions n(ca) in the starting composition provided in step (i) can be determined by standard analytical procedures well known in the art, e.g. acid base titration.

In the context of the present invention starting compositions comprising at least one polyunsaturated omega-3 or omega-6 fatty acid component may be any compositions containing substantial amounts of at least one polyunsaturated omega-3 or omega-6 fatty acid component, wherein each type (i.e. molecular species) of free omega-3 or omega-6 PUFA (with “free” indicating the presence of a free carboxylic acid function) constitutes a different polyunsaturated omega-3 or omega-6 fatty acid component. Such compositions may further comprise other naturally occurring fatty acids in free form. In addition, such compositions may further comprise constituents that by themselves are solid, liquid or gaseous at room temperature and standard atmospheric pressure. Corresponding liquid constituents include constituents that can easily be removed by evaporation and could thus be considered as volatile constituents as well as constituents that are difficult to remove by evaporation and could thus be considered as non-volatile constituents. In the present context gaseous constituents are considered as volatile constituents. Typical volatile constituents are water, alcohols and supercritical carbon dioxide.

Accordingly, typical starting compositions, without taking account for volatile constituents, have a PUFA-content (i.e. the total content of one or more free polyunsaturated omega-3 or omega-6 fatty acids) of at least 25 wt %, up to 75 wt % of other naturally occurring fatty acids in free form, and up to 5 wt % of other constituents that by themselves are solid or liquid at room temperature and standard atmospheric pressure. However, higher grades of polyunsaturated omega-3 or omega-6 fatty acids can be obtained by purification of the respective starting materials. In a preferred embodiment of the present invention starting compositions, without taking account for volatile constituents, have a PUFA-content (i.e. the total content of one or more free polyunsaturated omega-3 or omega-6 fatty acids) of at least 50 wt %, up to 50 wt % of other naturally occurring fatty acids in free form, and up to 5 wt % of other constituents that by themselves are solid or liquid at room temperature and standard atmospheric pressure. In another preferred embodiment of the present invention starting compositions, without taking account for volatile constituents, have a PUFA-content (i.e. the total content of one or more free polyunsaturated omega-3 or omega-6 fatty acids) of at least 75 wt %, up to 25 wt % of other naturally occurring fatty acids in free form, and up to 5 wt % of other constituents that by themselves are solid or liquid at room temperature and standard atmospheric pressure. In another preferred embodiment of the present invention starting compositions, without taking account for volatile constituents, have a PUFA-content (i.e. the total content of one or more free polyunsaturated omega-3 or omega-6 fatty acids) of at least 90 wt %, up to 10 wt % of other naturally occurring fatty acids in free form, and up to 5 wt % of other constituents that by themselves are solid or liquid at room temperature and standard atmospheric pressure. In another preferred embodiment of the present invention starting compositions, without taking account for volatile constituents, have a PUFA-content (i.e. the total content of one or more free polyunsaturated omega-3 or omega-6 fatty acids) of at least 90 wt %, up to 10 wt % of other naturally occurring fatty acids in free form, and up to 1 wt % of other constituents that by themselves are solid or liquid at room temperature and standard atmospheric pressure.

The counter ion composition provided in step (ii) of the process of the present invention is a composition comprising substantial amounts of a counter ion. This composition may further comprise constituents that by themselves are solid, liquid or gaseous at room temperature and standard atmospheric pressure. Corresponding liquid constituents include constituents that can easily be removed by evaporation and could thus be considered as volatile constituents as well as constituents that are difficult to remove by evaporation and could thus be considered as non-volatile constituents. In the present context gaseous constituents are considered as volatile constituents. Typical volatile constituents are water, alcohols and supercritical carbon dioxide. Typical lysine compositions contain at least 95 wt %, 97 wt %, 98 wt %, or 99 wt % of free lysine, without taking account for volatile constituents. Preferred lysine compositions contain at least 98 wt % of free lysine, without taking account for volatile constituents.

Spray granulation using solutions of omega salts is a specialized process involving more than one solvent and a complex set of parameters controlling the product properties. It was found during the experimentation, that there exists a meaningful correlation between the processability and the process parameters, which although cannot be generalized for a wide range of products, is specifically applicable for the omega salt spray granulation process. A mathematical formula was derived using the following factors: a) Average bed temperature during the omega salt spray granulation process, b) cubic root of average atomization pressure used and c) the cubic root of scale of operation/batch size. The derived mathematical formula for estimating the processability during the omega salt spray granulation process by determining a process factor (PF) is as mentioned below:

$$PF = \frac{( 3 \sqrt{S} \div T ) \times 100}{3 \sqrt{A}}$$

wherein S is the batch size in kg, T is the average bed temperature in ° C. and A is the average atomization pressure in bar.

Therefore, in an advantageous configuration of the present invention, the spray granulation is performed at an average bed temperature (T) of between 50° C. and 90° C., preferably between 50° C. and 80° C., at an average atomization pressure (A) between 0.5 and 10 bar, and the process factor is higher than 1.6, preferably between 1.6 and 10.0, wherein the process factor (PF) is defined as:

$$PF = \frac{( 3 \sqrt{S} \div T ) \times 100}{3 \sqrt{A}}$$

and wherein S is the batch size in kg, T is the average bed temperature in ° C. and A is the average atomization pressure in bar. For the continuous spray granulation process, the batch size S is the amount of solids present in the process chamber during processing.

In a preferred configuration, the granulation process is selected from spray granulation, dry granulation, slugging, planetary mixing granulation, high shear granulation, melt granulation and top spray granulation and from batch spray-granulation and continuous spray granulation as well as modified forms.

In a preferred configuration, the granulation process is selected from spray granulation, top spray granulation and from batch spray-granulation and continuous spray granulation as well as modified forms.

It is preferred, when the granulation is carried out in the presence of one or more excipients selected from diluents, binders, flow promoters, lubricants, plasticizers.

In a preferred configuration, the counter ion is a basic amine, preferably chosen from lysine, arginine, ornithine, choline, or a counter ion selected from magnesium (Mg²⁺) and potassium (K⁺), or mixtures thereof.

It is further preferred to use basic amines as counter ions selected from lysine, arginine and ornithine or a counter ion selected form magnesium (Mg²⁺) and potassium (K⁺).

It is particularly preferred, when L-lysine or a mixture of L-lysine and L-arginine are used as counter ions and that the ratio between L-lysine and L-arginine is between 10:1 and 1:1.

In preferred embodiments of the present invention, without accounting for volatile constituents, starting compositions contain mostly free PUFAs and other naturally occurring fatty acids in free form and counter ion compositions contain mostly free basic amine, preferably lysine or arginine, thus yielding product compositions mostly consisting of salts of lysine or arginine with PUFAs and other naturally occurring fatty acids.

In step (iii) of the process of the present invention starting composition and counter ion composition are combined. Combining can be achieved by any means allowing formation of a product composition comprising at least one salt of a cation with an anion derived from a polyunsaturated omega-3 or omega-6 fatty acid. Accordingly, a typical way of combining starting composition and counter ion composition would be admixing aqueous, aqueous-alcoholic or alcoholic solutions of each and removing the solvent subsequently. Alternatively, depending on the remaining constituents of the compositions, it may not be necessary to add solvents but could be sufficient to combine both compositions directly. In the context of the present invention a preferred way of combining both compositions is admixing aqueous, aqueous-alcoholic or alcoholic solutions of each and removing the solvent subsequently.

In the context of the present invention a cation derived from a basic amine selected from lysine, arginine, ornithine, choline, or mixtures thereof is a cation obtained by protonation of lysine, arginine, ornithine, choline, or mixtures thereof.

In the context of the present invention an anion derived from a polyunsaturated omega-3 or omega-6 fatty acid is an anion obtained by deprotonation of a polyunsaturated omega-3 or omega-6 fatty acid.

Accordingly, in preferred embodiments of the present invention, starting composition in step (i) and lysine composition in step (ii) are provided in such a manner that at least sp wt % of the product composition consist of one or more salts of cations derived from lysine with anions derived from one or more polyunsaturated omega-3 or omega-6 fatty acids and other naturally occurring fatty acids, wherein sp is selected from 50, 60, 70, 80, 90, 95, 97, 98, 99, 100.

In a further preferred configuration, the source for omega-3 or omega-6 fatty acids is chosen from at least one of the following: fish oil, squid oil, krill oil, linseed oil, borage seed oil, algal oil, hemp seed oil, rapeseed oil, flaxseed oil, canola oil, soybean oil.

The present invention further comprises particles obtainable by a process as described above.

The present invention further comprises particles comprising of one or more salts of cations derived from a counter ion with anions derived from one or more polyunsaturated omega-3 or omega-6 fatty acids obtainable by a granulation process, with a particle size distribution curve exhibiting at least two of the following properties:
A. D90 is between 350 μm and 1500 μm;
B. In multimodal curves, the tallest peak has a peak intensity in the range of 200 μm to 1500 μm, wherein the intensity (as measured on Y axis) of the second tallest peak is not more than 50% of the tallest peak;
C. In multimodal curves, the intensity difference (as measured using Y axis value) between the tallest and the second tallest peak is equal to or less than 30%, and the second tallest peak has the highest intensity in the range of 400 μm to 1500 μm, wherein the trough intensity on Y scale between above two peaks is more that 25% of the tallest peak;
D. Base of the tallest peak in the PSD curve (as measured by difference in microns between the two lowest points of the peak on ‘Y’ axis) is at least 400 μm wide by absolute value.

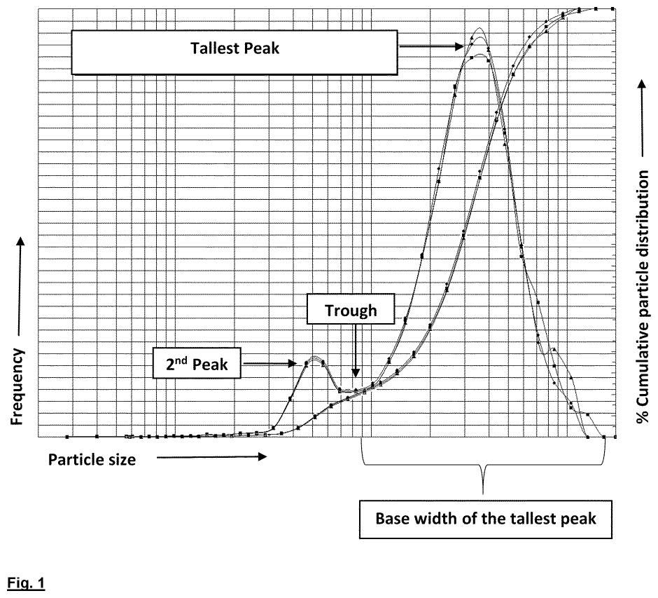

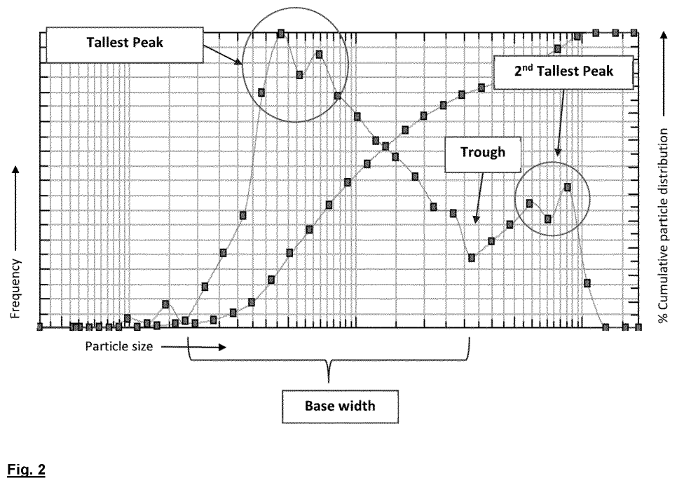

According to the present invention, a particle size distribution (PSD) curve shows the distribution of the particle size of a mixture of particles, where the particle size is shown on the X-axis and the respective cumulative percentage is shown on the Y-axis. Such a particle size distribution curve and the acceptance criteria A to D are depicted in FIGS. 1 and 2, with the following definitions:
1st Tallest Peak: The tallest curve in the PSD graph as measured on Y-axis.
2nd Tallest peak: The second tallest curve as compared to the 1st tallest peak in the PSD graph as measured on Y-axis.
Intensity difference: The curve intensity difference between 1st tallest and 2nd tallest curve in the PSD graph as measured on Y-axis
Base width: The value on X-axis (in microns) calculated by drawing perpendiculars from the lowest two points or troughes on the two sides of the peak.
Trough intensity: The lowest point on the Y-axis existing between the two peaks

To define the distribution width three values on the X-axis are used, the D10, D50, and D90 value. For particle size distributions the median is called the D50 and is the size in microns that splits the distribution with half above and half below this diameter. Similarly, 90 percent of the distribution lies below the D90, and 10 percent of the population lies below the D10.

In a preferred configuration, the counter ion is a basic amine, preferably chosen from lysine, arginine, ornithine, choline, or a counter ion selected from magnesium (Mg2+) and potassium (K+), or mixtures thereof.

In a preferred embodiment, the counter ion composition for the particles is provided in such manner that the ratio of the amount of carboxylic acid functions in the starting composition and the amount of counter ions is in a range of 1:0.5 to 1:2 (carboxylic acid functions:counter ions) on molar basis. In other words, this means that the starting omega-3 or omega-6 fatty acid component and the counter ion composition shall be provided in equimolar quantities to facilitate quantitative salt formation.

In a preferred embodiment, the counter ion composition is provided in such a manner that the ratio R=n(ca)/n(ci) of the amount of carboxylic acid functions n(ca) in the starting composition and the total amount of free counter ion n(ci) in the counter ion composition is in a range selected from 0.9<R<1.1, 0.95<R<1.05, 0.98<R<1.02. In a particularly preferred embodiment R is in the range 0.98 <R<1.02. The amount of carboxylic acid functions n(ca) in the starting composition can be determined by standard analytical procedures well known in the art, e.g. acid base titration.

It is preferred, wherein the granulation process is selected from spray granulation, dry granulation, slugging, planetary mixing granulation, high shear granulation, melt granulation and top spray granulation and from batch spray-granulation and continuous spray granulation as well as modified forms, preferably selected from spray granulation, top spray granulation and from batch spray-granulation and continuous spray granulation as well as modified forms.

It is preferred, when the granulation is carried out in the presence of one or more excipients selected from diluents, binders, flow promoters, lubricants.

A further subject of the present invention is the use of particles according to the present invention for the manufacture of food products comprising polyunsaturated omega-3 or omega-6 fatty acids.

In the context of the present invention food products comprise but are not limited to baked goods, vitamin supplements, diet supplements, powdered drinks, doughs, batters, baked food items including e.g. cakes, cheesecakes, pies, cupcakes, cookies, bars, breads, rolls, biscuits, muffins, pastries, scones, and croutons; liquid food products e.g. beverages, energy drinks, infant formula, liquid meals, fruit juices, multivitamin syrups, meal replacers, medicinal foods, and syrups; semi-solid food products such as baby food, yogurt, cheese, cereal, pancake mixes; food bars including energy bars; processed meats; ice creams; frozen desserts; frozen yogurts; waffle mixes; salad dressings; and replacement egg mixes; and further cookies, crackers, sweet goods, snacks, pies, granola/snack bars, and toaster pastries; salted snacks such as potato chips, corn chips, tortilla chips, extruded snacks, popcorn, pretzels, potato crisps, and nuts; specialty snacks such as dips, dried fruit snacks, meat snacks, pork rinds, health food bars and rice/corn cakes; confectionary snacks such as candy; instant food products, such as instant noodles, instant soup cubes or granulates.

A further subject of the present invention is the use of particles according to the present invention for the manufacture of nutritional products comprising polyunsaturated omega-3 or omega-6 fatty acids.

In the context of the present invention nutritional products comprise any type of nutraceutical, nutrient or dietary supplement, e.g. for supplementing vitamins, minerals, fiber, fatty acids, or amino acids.

A further subject of the present invention is the use of particles according to the present invention for the manufacture of pharmaceutical products comprising polyunsaturated omega-3 or omega-6 fatty acids.

In the context of the present invention the pharmaceutical product can further comprise a pharmaceutically acceptable excipient as well as further pharmaceutically active agents including for example cholesterol-lowering agents such as statins, anti-hypertensive agents, anti-diabetic agents, anti-dementia agents, anti-depressants, anti-obesity agents, appetite suppressants and agents to enhance memory and/or cognitive function.

A solid oral dosage form prepared from particles according to the present invention is also a subject of the present invention, wherein the solid oral dosage form is selected from tablets, granules or capsules.

In a preferred configuration, the omega-3 fatty acid component is selected from EPA or DHA. In a further preferred configuration, the omega-3 or omega-6 fatty acid salt has an organic counter ion selected from lysine, arginine, ornithine, choline or magnesium (Mg2+), potassium (K+) and mixtures of the same.

In a preferred embodiment, the amount of polyunsaturated fatty acid is 65 weight % or less, preferably 60 weight % or less, more preferably between 40 and 55 weight-% with respect to the total weight of polyunsaturated fatty acid salt.

In an alternative configuration, the amount of polyunsaturated fatty acid is over 80%, preferably over 90%. Specifically, for the magnesium salt the content of polyunsaturated fatty acid may be over 90%, more specifically around 93%. In another specific embodiment, for the potassium salt, the amount of polyunsaturated fatty acid may be over 85%, more specifically around 89%.

In a preferred embodiment, the amount of polyunsaturated fatty acid salt in the tableting composition is 50 weight-% or less, preferably 40 weight-% or less, more preferably between 0.5 and 30 weight-%.

### EXAMPLES

### Comparative Examples 1-3

### Spray Drying Process

Process details for spray drying (C1-C3): PUFA lysine salts hydroethanolic solutions were prepared and spray dried using below mentioned process parameters (table 1).

TABLE 1
Spray drying process parameters

| Process parameters | C-1 | C-2 | C-3 |
|---|---|---|---|
| Batch size (g) | 2243 | 2752 | 100 |
| Gas inlet temperature (° C.) | 170 | 170 | 52-57 |
| Aver. atomization air pressure (bar) | 6 | 6 | 10 |

TABLE 2
Spray drying process - granules characterization

| Example | C-1 | C-2 | C-3 |
|---|---|---|---|
| Bulk Density (g/cc) | — | — | 0.294 |
| Tapped density (g/cc) | — | — | 0.408 |
| Compressibility index (%) | — | — | 27.94 |
| Angle of repose | — | — | 42.55 |

PSD data

| | | | |
|---|---|---|---|
| Avg D90 (μm) | 82.332 | 95.813 | 60.64 |
| Type of PSD curve | Monomodal | Monomodal | Multimodal |
| Mean tallest peak intensity | 7.96, 7.45 | 7.99, 7.78 | ~2.32 |
| in the PSD curve (Y-axis) | | | |
| Tallest peak point in the PSD | ~40 | 40-50 | ~35-40 |
| curve (X-axis, μm) | | | |
| 2^{nd} tallest peak | — | — | 0.86 |
| in the PSD curve | | | |
| (Y-axis) | | | |
| 2^{nd} tallest peak | — | — | 15 |
| point in the PSD | | | |
| curve (X-axis, μm) | | | |
| Intensity difference | — | — | 62.93 |
| (Y-axis) of 2^{nd} | | | |
| tallest peak as compared to | | | |
| tallest peak (limit NMT 50%) | | | |
| Lowest trough between | — | — | 0.15-0.17 |
| the tallest | | | |
| and 2^{nd} tallest peak | | | |
| Trough intensity wrt to | — | — | 6.90 |
| tallest peak | | | |
| (limit more than 25%) | | | |
| Base width of the | ~110 | ~120 | 282.2 |
| tallest peak | | | |
| (μm) | | | |

The products could not be processed on a tableting machine, due to bad flow properties. The characterization of the granules is summarized in table 2. The criteria A to D as defined above were not met.

### Comparative Examples 4-6

### Spray Granulation with Recirculation of Fines

Process details for spray granulation (C4-C5): PUFA lysine salts hydroethanolic solutions were prepared and spray granulated using below mentioned process parameters (table 3). For comparative example C-6, PUFA lysine salt was granulated with a Rapid mixer granulator (CPM RMG-10, Chamunda Pharma Machinary Pvt. Ltd.).

TABLE 3
Process parameters for comparative examples C-4 to C-6

| Process parameters | C-4 | C-5 | C-6 |
|---|---|---|---|
| Batch size (g) | 1000 | 1500 | 500 |
| Inlet air temperature (° C.) | 115 | 115 | 40 |
| Average bed temperature (° C.) | 68 | 72 | — |
| Atomization air pressure (bar) | 1 | 1 | — |
| Process factor | 1.47 | 1.59 | — |

TABLE 4
Spray granulation process - granules characterization

| Example | C-4 | C-5 | C-6 |
|---|---|---|---|
| Bulk Density (g/cc) | 0.403 | 0.417 | 0.403 |
| Tapped density (g/cc) | 0.521 | 0.545 | 0.521 |
| Compressibility index (%) | 22.581 | 23.577 | 22.581 |
| Avg D90 (μm) | 602.30 | 309.83 | 400-595 |
| Type of PSD curve | Multimodal | Multimodal | Monomodal |
| Mean tallest peak intensity | ~1.6 | ~1.3 | |
| in the PSD curve (Y-axis) | | | |
| Tallest peak point in the | 45-50 | 40-60 | |
| PSD curve (X-axis, μm) | | | |
| 2nd tallest peak in | ~0.87 | ~1.16 | |
| the PSD curve (Y-axis) | | | |
| 2nd tallest peak point in | 500-800 | 100-200 | |
| the PSD curve (X-axis, μm) | | | |
| Intensity difference (Y-axis) | 45.63 | 10.77 | |
| of 2nd tallest peak as | | | |
| compared to tallest | | | |
| peak (limit NMT 50%) | | | |
| Lowest trough between | 0.2-0.3 | 0.825-0.925 | |
| the tallest | | | |
| and 2nd tallest peak | | | |
| Trough intensity wrt | 15.63 | 67.31 | |
| to tallest peak | | | |
| (limit more than 25%) | | | |
| Base width of the | 200 | 70 | ~800 |
| tallest peak | | | |
| (μm) | | | |

The products could not be processed on a tableting machine, due to bad flow properties or issues related to sticking of tablets on tooling or both. The characterization of the granules is summarized in table 4. The criteria A to D as defined above were not met for C4 and C5.

### Examples 1-5

### Spray Granulation with Recirculation of Fines (Inventive)

Process details for spray granulation: PUFA lysine salts hydroethanolic solutions were prepared and spray granulated using below mentioned process parameters (see table 5).

TABLE 5
Spray granulation process parameters

| Process parameters | 1 | 2 | 3 | 4 | 5 |
|---|---|---|---|---|---|
| Batch size (g) | 1000 | 1000 | 1000 | 750 | 1500 |
| Average bed | 61 | 64 | 59 | 58 | 60 |
| temperature (° C.) | | | | | |
| Aver. atomization | 1 | 0.6 | 0.6 | 0.6 | 0.6 |
| pressure (bar) | | | | | |
| Process factor (P.F.) | 1.64 | 1.85 | 2.01 | 1.86 | 2.26 |

TABLE 6
Spray granulation process - granules characterization

| Example | 1 | 2 | 3 | 4 | 5 |
|---|---|---|---|---|---|
| Bulk Density (g/cc) | 0.452 | 0.421 | 0.455 | 0.419 | 0.439 |
| Tapped density (g/cc) | 0.556 | 0.484 | 0.560 | 0.543 | 0.512 |
| Compressibility index (%) | 18.669 | 12.919 | 18.770 | 22.900 | 14.327 |

PSD data

| | | | | | |
|---|---|---|---|---|---|
| Avg D90 (μm) | 891.75 | 612.89 | 1005.00 | 918.36 | 1103.08 |
| Type of PSD curve | Multimodal | Multimodal | Multimodal | Multimodal | Multimodal |
| Mean tallest peak intensity in the | 1.2 | 1.67 | 1.73 | 1.39 | 1.74 |
| PSD curve (Y-axis) | | | | | |
| Tallest peak point in the PSD | 50-60 | 300-400 | 800-900 | 700-900 | 700-800 |
| curve (X-axis, μm) | | | | | |
| 2^{nd} tallest peak in the PSD curve | ~0.711 | 0.33 | 0.79 | 1.03 | 0.433 |
| (Y-axis) | | | | | |
| 2^{nd} tallest peak point in the PSD | 800-900 | 44-60 | 46-52 | 50-58 | 50 |
| curve (X-axis, μm) | | | | | |
| Intensity difference (Y-axis) of 2^{nd} | 40.75 | 80.23 | 54.34 | 25.90 | 75.11 |
| tallest peak as compared to tallest | | | | | |
| peak (limit NMT 50%) | | | | | |
| Lowest trough between the tallest | 0.125-0.275 | 0.2 | 0.15 | 0.2-0.225 | 0.09-0.1 |
| and 2^{nd} tallest peak | | | | | |
| Trough intensity wrt to tallest peak | 16.67 | 11.98 | 8.67 | 15.29 | 5.46 |
| (limit more than 25%) | | | | | |
| Base width of the tallest peak | 300 | ~1400 | ~1300 | ~1300 | ~1300 |
| (μm) | | | | | |
| Workability (flow) on tableting | No | Yes | Yes | Yes | Yes |
| machine | | | | | |
| Remarks (acceptance | Passed | Passed | Passed | Passed | Passed |
| criteria) | A, B | A, D | A, D | A, B, D | A, D |

The characterization of the granules is shown in table 6. The acceptance criteria A to D as defined above were analyzed: according to the present invention, the particle size distribution curve shall exhibit at least two of the following properties:
A. D90 is between 400 μm and 1500 μm;
B. In multimodal curves, the tallest peak has a peak intensity in the range of 200 μm to 1500 μm, wherein the intensity (as measured on Y axis) of second tallest peak is not more than 50% of the tallest peak;
C. In multimodal curves, the intensity difference (as measured using Y axis value) between the tallest and the second tallest peak is equal to or less than 30%, and the second tallest peak has the highest intensity in the range of 400 μm to 1500 μm, wherein the trough intensity on Y scale between above two peaks is more that 25% of the tallest peak;
D. Base of the tallest peak in the PSD curve (as measured by difference in microns between the two lowest points of the peak on Y axis) is at least 400 μm wide by absolute value.

In all the examples, particles were produced, which fulfilled at least two of the listed acceptance criteria A to D and workability on the tableting machine was possible.

### Example 6

### Granulation Using Top Spray Granulation (Inventive)

PUFA lysine salt was granulated with water using top spray granulator using below mentioned process parameters (table 7).

For the experiments using planetary mixer 500 g of lysine-salt of omega-3 fatty acid was granulated for 2 mins with 22-25 g of purified water. The wet granules were dried to a LOD of <2.5% and sized to obtained desired particle size.

TABLE 7
Spray granulation process parameters

| Process parameters | 6 |
|---|---|
| Granulation technique | Top spray |
| | granulation |
| Batch size (g) | 800 |
| Residual moisture (%) | 0.5 |
| Inlet air temperature (° C.) | 50-65 |
| Average bed temperature (° C.) | 35-45 |
| Aver. atomization pressure (bar) | 1 |
| Process factor (P.F.) | 2.32 |

TABLE 8
Spray granulation process - granules characterization

| Example | 6 |
|---|---|
| Bulk Density (g/cc) | 0.401 |
| Tapped density (g/cc) | 0.457 |
| Compressibility index (%) | 12.297 |

PSD data

| | |
|---|---|
| Avg D90 (μm) | 585.69 |
| Type of PSD curve | Monomodal |
| Mean tallest peak intensity in the | 1.52 |
| PSD curve (Y-axis) | |
| Tallest peak point in the PSD | 200-300 |
| curve (X-axis, μm) | |
| 2nd tallest peak in the PSD curve | — |
| (Y-axis) | |
| 2nd tallest peak point in the PSD | — |
| curve (X-axis, μm) | |
| Intensity difference (Y-axis) of 2nd | — |
| tallest peak as compared to tallest | |
| peak (limit NMT 50%) | |
| Lowest trough between the tallest | — |
| and 2nd tallest peak | |
| Trough intensity wrt to tallest peak | — |
| (limit more than 25%) | |
| Base width of the tallest peak | ~1500 |
| (μm) | |
| Workability on tableting machine | Yes |
| Remarks (acceptance criteria) | Passed A, D |

The characterization of the granules is shown in table 8. The acceptance criteria A to D as defined above were analyzed.

In all the examples, particles were produced, which fulfilled at least two of the listed acceptance criteria A to D and workability on the tableting machine was possible.

### Examples 7-9

### Spray Granulation with Top Granulation Technique (Inventive)

PUFA lysine salt was granulated with water using top spray granulator using below mentioned process parameters (table 9).

TABLE 9
Top spray granulation process parameters

| Process parameters | 7 | 8 | 9 |
|---|---|---|---|
| Batch size (g) | 4500 | 4500 | 10500 |
| Average bed temperature (° C.) | 62 | 68 | 68 |
| Aver. atomization air pressure (bar) | 1.3-1.8 | 2.5 | 2.5 |
| Process factor (P.F.) | 2.33 | 1.79 | 2.37 |

TABLE 10
Spray granulation process - granules characterization

| Example | 7 | 8 | 9 |
|---|---|---|---|
| Bulk Density (g/cc) | 0.489 | 0.459 | 0.454 |
| Tapped density (g/cc) | 0.588 | 0.565 | 0.557 |
| Compressibility index (%) | 16.560 | 18.587 | 18.523 |

PSD data

| | | | |
|---|---|---|---|
| Avg D90 (μm) | 648.41 | 423.24 | 504.507 |
| Type of PSD curve | Multimodal | Monomodal | Multimodal |
| Mean tallest peak | ~1.075 | ~0.99 | ~1.01 |
| intensity in the | | | |
| PSD curve (Y-axis) | | | |
| Tallest peak point | 40-50 | 70 | 70 |
| in the PSD | | | |
| curve (X-axis, μm) | | | |
| 2nd tallest peak in | 0.635 | — | 0.342 |
| the PSD curve | | | |
| (Y-axis) | | | |
| 2nd tallest peak point | ~700-890 | — | ~1500 |
| in the PSD | | | |
| curve (X-axis, μm) | | | |
| Intensity difference | 40.93 | — | 66.14 |
| (Y-axis) of 2nd | | | |
| tallest peak as | | | |
| compared to tallest | | | |
| peak (limit NMT 50%) | | | |
| Lowest trough between | 0.25-0.30 | — | 0.175-0.325 |
| the tallest | | | |
| and 2nd tallest peak | | | |
| Trough intensity wrt | 25.58 | — | 24.75 |
| to tallest peak | | | |
| (limit more than 25%) | | | |
| Base width of the | 240 | ~1500 | 850 |
| tallest peak | | | |
| (μm) | | | |
| Workability on | Yes | Yes | Yes |
| tableting machine | | | |
| Remarks | Passed A, C | Passed A, D | Passed A, D |

The characterization of the granules is shown in table 10. The acceptance criteria A to D as defined above were analyzed.

In all the examples, particles were produced, which fulfilled at least two of the listed acceptance criteria A to D and workability on the tableting machine was possible.

### Example 10

### Tableting Trials

PUFA salts were prepared using spray granulation with recirculation of fines (as described above for comparative example C-4) and using spray granulation according to the inventive example 2 and formulated as shown in table 11 with tableting excipients for tableting trials.

TABLE 11
Compositions for tableting trials

| Component (mg) | Use | C-4 (comparative) | 2 |
|---|---|---|---|
| PUFA lysine salts | | 400.00 | 400.00 |
| Prosolv Easy tab Nutra | Filler | 356.00 | 356.00 |
| Aerosil 200 P | Glidant | 8.00 | 8.00 |
| Croscarmellose sodium | Disintegrant | 16.00 | 16.00 |
| Magnesium stearate | Lubricant | 20.00 | 20.00 |

TABLE 12
Characterization of tablets

| | C4 (comparative) | 2 |
|---|---|---|
| Target tablet weight (mg) | 800.00 | 800.00 |
| Tablet weight (mg) | 799-810 | 799-805 |
| Tablet thickness (mm) | 5.77-5.80 | 5.62-5.65 |
| Hardness (N) | 73-77 | 83-88 |
| Friability (%) | 0.2133 | 0.246 |
| Workability (flow) on tableting machine | No | Yes |

The results of the tableting trials are summarized in table 12. Workability on tableting machine was only possible with granules produced according to the present invention.

### Examples 11-13

### Spray Granulation Using Different PUFA Salts (Inventive)

For the inventive examples 11 and 12, the PUFA potassium salts/PUFA ornithine salts solution (50% w/w) in 50% hydroethanolic and spray granulated using below mentioned process parameters. For the inventive example 13, PUFA lysine salts solution (50% w/w) were prepared in a hydroethanolic solution and spray granulated using below mentioned process parameter in a continuous fluidized bed granulator with a sieve-grinding cycle (see table 13).

TABLE 13
Spray granulation process parameters

| Process parameters | 11 | 12 | 13 |
|---|---|---|---|
| Batch size (g) | 1300 | 1300 | 16600 |
| Average bed temperature (° C.) | 55 | 53 | 60 |
| Aver. atomization pressure (bar) | 1.2 | 1.2 | 2.0 |
| Process factor (P.F.) | 1.87 | 1.94 | 3.37 |

TABLE 14
Spray granulation process - granules characterization

| Example | 11 | 12 | 13 |
|---|---|---|---|
| Bulk Density (g/cc) | 0.439 | 0.402 | 0.39 |
| Tapped density (g/cc) | 0.491 | 0.494 | 0.43 |
| Compressibility index (%) | 10.59 | 18.62 | 8.11 |

PSD data

| | | | |
|---|---|---|---|
| Avg D90 (μm) | 892.3 | 873.7 | 805.9 |
| Type of PSD curve | Monomodal | Monomodal | Monomodal |
| Mean tallest peak | — | — | — |
| intensity in the | | | |
| PSD curve (Y-axis) | | | |
| Tallest peak point | 541.9 | 594.9 | 594.9 |
| in the PSD | | | |
| curve (X-axis, μm) | | | |
| 2nd tallest peak | — | — | — |
| in the PSD curve | | | |
| (Y-axis) | | | |
| 2nd tallest peak | — | — | — |
| point in the PSD | | | |
| curve (X-axis, μm) | | | |
| Intensity difference | — | — | — |
| (Y-axis) of 2nd | | | |
| tallest peak as | | | |
| compared to tallest | | | |
| peak (limit NMT 50%) | | | |
| Lowest trough between | — | — | — |
| the tallest | | | |
| and 2nd tallest peak | | | |
| Trough intensity wrt | — | — | — |
| to tallest peak | | | |
| (limit more than 25%) | | | |
| Base width of the | 1436 | 1179 | 1051 |
| tallest peak | | | |
| (μm) | | | |
| Workability on | Yes | Yes | Yes |
| tableting machine | | | |
| Remarks | Passed A, D | Passed A, D | Passed A, D |

### Tableting Trials:

PUFA salts were prepared as described above for inventive example 11-13 and were formulated as shown below. The tableting composition is summarized in table 15 and the results of the tableting trials are summarized in table 16.

TABLE 15
Compositions for tableting trials

| Component (mg) | 11 | 12 | 13 |
|---|---|---|---|
| PUFA potassium salt | 243 | — | — |
| PUFA ornithine salt | — | 243 | — |
| PUFA lysine salt | — | — | 243 |
| Microcrystalline cellulose | 550.8 | 550.8 | 550.8 |
| (Avicel 200) | | | |
| Croscarmellose sdium (Ac-di-sol) | 16.2 | 16.2 | 16.2 |
| Total (tablet weight) | 810 | 810 | 810 |

TABLE 16
Characterization of tablets

| | 11 | 12 | 13 |
|---|---|---|---|
| Target tablet weight (mg) | 810 | 810 | 810 |
| Actual tablet weight (mg) | 793-820 | 798-814 | 804-814 |
| Tablet thickness (mm) | 5.91-6.03 | 6.67-6.72 | 6.92-6.98 |
| Hardness (N) | 47-55 | 71-77 | 63-76 |
| Friability (%) | 0.06 | 0.33 | 0.59 |
| Workability (flow) on tableting | Yes | Yes | Yes |
| machine | | | |

### Scanning Electron Microcopy (SEM) Studies:

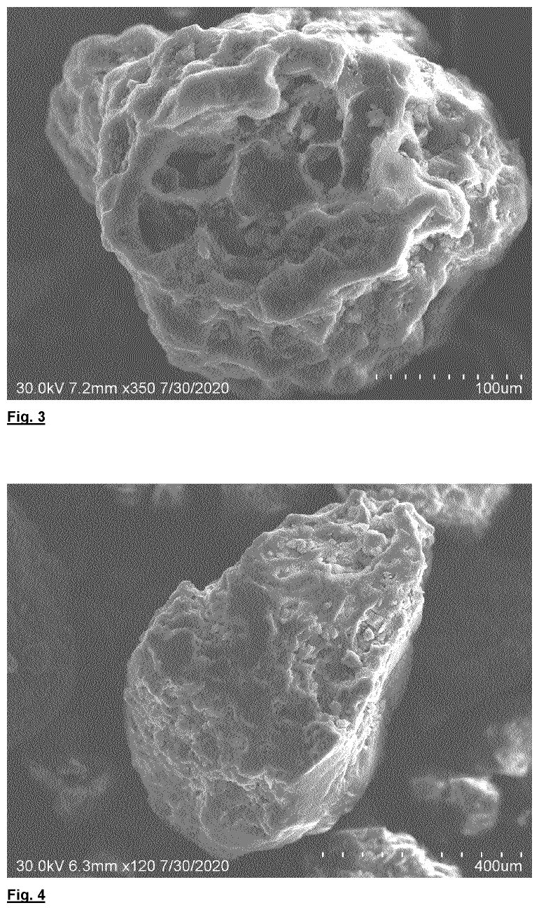

PUFA salts prepared as described in inventive example 13 (PUFA lysine salts, prepared by continuous granulation) and comparative example C6 (PUFA lysine salts, prepared by rapid mixer granulation) were evaluated using SEM to understand particle surface characteristics (internal structure). The results are shown in FIG. 3 and FIG. 4.

As shown in FIG. 3, the internal structure of spray granulated PUFA salt prepared according to inventive example I-13 has a highly porous nature. In contrast to this, for RMG granulated PUFA salt prepared according to comparative example C-6, such porous structure was not seen (FIG. 4). Instead it was more rigid, thus less preferred for tableting operations.

### Exposure to High Humidity on the PUFA Salt Granules Prepared Using Different Methods:

PUFA salts from inventive example 13 (PUFA lysine salts, prepared by continuous granulation) and comparative example C6 (PUFA lysine salts, prepared by rapid mixer granulation) were exposed to 40° C./75% relative humidity (RH) conditions for 1 hour and observed under microscope in order to understand the sensitivity of these materials while handling during tableting operations.

After exposure of the samples to 40° C./75% relative humidity (RH) conditions for 1 hour, the surface of continuous spray granulated PUFA lysine salt showed no appreciable changes due to high temperature and humidity exposure. In contrast to this, the surface of rapid mixer granulated PUFA lysine salt turned sticky and oily on exposure difficult to process further for tableting.

1. A process for granulating a polyunsaturated fatty acid salt, comprising:
i. providing a starting composition comprising at least one polyunsaturated omega-3 or omega-6 fatty acid component;
ii. providing a counter ion composition;
iii. admixing an aqueous, aqueous-alcoholic or alcoholic solution comprising the starting composition and the counter ion composition to form an admixture; and
iv. subjecting the admixture to granulation in a fluidized bed, thereby forming a solid product composition comprising at least one salt of a cation derived from the counter ion with an anion derived from a polyunsaturated omega-3 or omega-6 fatty acid,
wherein a ratio of an amount of carboxylic acid functions in the starting composition to an amount of counter ions provided is in a range of 1:0.5 to 1:2 (carboxylic acid functions:counter ions) on molar basis.

2. The process of claim 1, wherein the granulation is performed at an average bed temperature (T) of 50° C. to 90° C., at an average atomization pressure (A) of 0.5 to 10 bar, and with a process factor of greater than 1.6 to 10.0, wherein the process factor (PF) is defined as:
$$PF = \frac{( 3 \sqrt{S} \div T ) \times 100}{3 \sqrt{A}}$$
wherein S is a batch size in kg, T is the average bed temperature in ° C., and A is the average atomization pressure in bar.

3. The process of claim 1, wherein the granulation is selected from the group consisting of spray granulation, dry granulation, slugging, planetary mixing granulation, high shear granulation, melt granulation, and top spray granulation, the granulation being batch granulation, continuous granulation, or modified forms thereof.

4. The process of claim 1, wherein the counter ion is a basic amine selected from the group consisting of lysine, arginine, ornithine, and choline, or a metal ion selected from magnesium (Mg²⁺), potassium (K⁺), and mixtures thereof.

5. The process of claim 1, wherein the counter ion is L-lysine or a mixture of L-lysine and L-arginine having a ratio of L-lysine to L-arginine of 10:1 to 1:1.

6. The process of claim 1, wherein the omega-3 or omega-6 fatty acid is derived from a fatty acid source which is at least one selected from the group consisting of: fish oil, squid oil, krill oil, linseed oil, borage seed oil, algal oil, hemp seed oil, rapeseed oil, flaxseed oil, canola oil, and soybean oil.

7. Particles formed by the process of claim 1.

8. Particles comprising one or more salts of cations derived from a counter ion with anions derived from one or more polyunsaturated omega-3 or omega-6 fatty acids, the particles obtainable by a granulation process with and having a particle size distribution curve exhibiting at least two of the following properties:
A. D90 is between 350 μm and 1500 μm,
B. In multimodal curves, a tallest peak has a peak intensity in the range of 200 μm to 1500 μm, wherein the peak intensity (as measured on Y axis) of a second tallest peak is not more than 50% of the tallest peak;
C. In multimodal curves, an intensity difference (as measured using Y axis value) between the tallest and the second tallest peak is equal to or less than 30%, and the second tallest peak has the highest peak intensity in the range of 400 μm to 1500 μm, wherein a trough intensity on Y scale between above two peaks is more that 25% of the tallest peak; and
D. A base of the tallest peak in the PSD curve (as measured by difference in microns between the two lowest points of the peak on Y axis) is at least 400 μm wide by absolute value.

9. The particles of claim 8, wherein the counter ion is a basic amine, selected from the group consisting of lysine, arginine, ornithine, and choline, or a metal ion selected from magnesium (Mg²⁺), potassium (K⁺), and mixtures thereof.

10. The particles of claim 8, wherein a ratio of an amount of carboxylic acid functions to an amount of counter ions is in a range of 1:0.5 to 1:2 (carboxylic acid functions:counter ions) on molar basis.

11. The particles of claim 8, wherein the granulation is selected from the group consisting of spray granulation, dry granulation, slugging, planetary mixing granulation, high shear granulation, melt granulation, and top spray granulation, the granulation being batch granulation, continuous granulation, or modified forms thereof.

12. The particles of claim 8, wherein the granulation is carried out in the presence of one or more excipients selected from the group consisting of diluents, binders, flow promoters, lubricants, and plasticizers.

13. A method of manufacturing food products comprising polyunsaturated omega-3 or omega-6 fatty acids, wherein the polyunsaturated omega-3 or omega-6 fatty acids are present as the particles of claim 8.

14. A method of manufacturing nutritional products comprising polyunsaturated omega-3 or omega-6 fatty acids, wherein the polyunsaturated omega-3 or omega-6 fatty acids are present as the particles of claim 8.

15. A method of manufacturing pharmaceutical products comprising polyunsaturated omega-3 or omega-6 fatty acids, wherein the polyunsaturated omega-3 or omega-6 fatty acids are present as the particles of claim 8.

16. A solid oral dosage form comprising the particles of claim 8, wherein the solid oral dosage form is selected from tablets, granules or capsules.
